(19) United States
(12) Patent Application Publication
(10) Pub. No.: US 2003/0014773 A1
(43) Pub. Date: Jan. 16, 2003

(54) Cytoplasmic male sterility-based system for hybrid wheat plant and seed production

(76) Inventors: Ichiro Ohtsuka, Naka-gun (JP); Calvin F. Konzak, Pullman, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/163,076

(22) Filed: Jun. 4, 2002

Related U.S. Application Data
(60) Provisional application No. 60/295,957, filed on Jun. 4, 2001.

(51) Int. Cl. A01H 5/00; A01H 1/02

(52) U.S. Cl. 800/274; 800/320.3; 800/278

(57) ABSTRACT

The present invention relates to the production of hybrid wheat and triticale plants and seeds via the employment of genetically-controlled cytoplasmic male sterility (CMS) and genetically-controlled fertility restoration. The genetically-controlled cytoplasmic sterility mechanism according to the present system and method utilizes genes conferring CMS in the presence of Aegilops squarrosa cytoplasm, and for restoring fertility to wheat and triticale plants having Aegilops species cytoplasm. The invention also relates to the use of specific, induced mutant sources of tolerance to herbicides for destroying the seed producing ability of the male fertility restorer parent after pollination has completed, to achieve essentially 100% hybrid seed reproduction from a mixture of a CMS male sterile line and a pollen fertility restorer line.

This applications claims the benefit of U.S. Provisional Patent Application No. 60/295,957, filed Jun. 4, 2001, the disclosure of which is incorporated herein.

### BACKGROUND OF THE INVENTION

Wheat and triticale are used for the production of food, for the commercial processes leading to products for human consumption, for animal feedstuff production, for the development of industrial products, and other purposes. Generally, wheat and triticale are bred from regional, climatic adapted plant varieties that have the desired properties. Seeds are produced and distributed to farmers, who plant the seed for later harvest.

Wheat and triticale plant varieties can be either line varieties or hybrid varieties. Line varieties are generally homozygous in their genetic composition, having mostly identical gene alleles on the two haploid sets of chromosomes in their genomes. Hybrid varieties are largely heterozygous in their genetic composition, having different gene alleles for an undefined number of genes on each of the two haploid sets of chromosomes in their genomes. The heterozygosity of hybrid varieties, together with other undefined genetic effects, leads to a phenomenon called heterosis, which is exhibited as increased vigor and yield performance of the varieties, compared to the parental lines. Heterosis can often result in increased vigor and yield performance as compared to the best performing parental line.

Factors involved in the production of hybrid seed include controlled cross-pollination while limiting self-pollination, allowing sufficient pollen transfer, and retaining hybrid vigor and desirable characteristics in the progeny. Several methods have been proposed to limit self pollination (selling) of the parental lines. These methods include emasculation, chemically-induced male sterility, genetically-induced male sterility, cytoplasmic male sterility, day length incompatibility and self-incompatibility. For example, emasculation can be achieved manually or mechanically on tomato and maize, respectively. Emasculation is generally not applicable, however, to wheat and triticale due to flower architecture and scale(s) of production.

Chemically-induced male sterility has been used to make male sterile, female plants by application of a chemical hybridizing agent (CHA) or gametocide, such as proposed by Orr and Clifford (see, e.g., U.S. Pat. No. 4,569,688), or an agent such as the Monsanto gametocide, ‘Genesis’. The female parental line is typically sprayed with the CHA or gametocide to render it male sterile. The female parental line is planted in an area that is surrounded by the intended fertile male parental line. Alternatively, the parental lines can be planted in adjacent strips. The transfer of pollen by wind from fertile male plants to male sterile, female plants results in the production of hybrid seed, which can then be sold to the farmer. Unfortunately, the CHA N-formyl-3-carboxyazetidine (see U.S. Pat. No. 4,569,688) was found to be unsuitable due to a health hazard of this CHA. The Monsanto gametocide, ‘Genesis’, however, was cleared for commercial application, and was tested for the commercial production of hybrid seed.

Several factors have limited the use of CHA's and gametocides. One of these factors is the requirement to separately grow the fertile male and the male sterile, female parent plants in order to allow application of the CHA or gametocide to the female parent plants. As the hybrid seed from the female plants must be separately harvested from that of the male parent. The effectiveness of this method can be limited by wind-facilitated cross-pollination. Another factor, or limitation, is the frequently variable effect(s) of the CHA or gametocide on the induction of male sterility. A fourth factor is the cost of the application of CHA or gametocide to the plants to make them male sterile. These factors combined have made the use of CHA's or gametocides uneconomical.

Genetically-induced male sterility of a euplasmic parental line has been proposed. For example, one line would be male or female sterile due to the presence of certain nuclear genes. (See, e.g., International PCT Publication No. WO/98/51142; U.S. Pat. No. 5,633,441; European Patent Publication EP 0 455 665 B1.) The nuclear genes could be naturally-occurring, or induced by transformation-based genetic modification of the plant (see, e.g., U.S. Pat. No. 5,633,441; European Patent Publication EP 0 455 665 B1). However, no wheat or triticale hybrid seed production method utilizing nuclear genes for genetically-induced male sterility has been established on a commercial level so far, leading to the conclusion that maintenance of the male sterile female plants is too difficult or too costly.

Other methods for hybrid wheat production have been proposed to utilize cytoplasmically-controlled male sterility, or CMS. (See, e.g., Franckowiak et al., Crop Science 16:725-28 (1976).) By such methods, plants are rendered male sterile due to cytoplasm exchange, leading to alloplasmic plants, where incompatibility occurs between an alien cytoplasm and the nuclear genome. Several levels of incompatibility and male sterility have been observed, however, in alloplasmic wheat and triticale, depending on the specific cytoplasm and genomic composition of the nuclear genome. (See, e.g., Franckowiak et al., Crop Science 16:725-28 (1976); Maan and Kianian, Wheat Information Service 93:5-8 (2001); Maan and Kianian, Wheat Information Service 93:2731 (2001); Asakura et al., Genome 43:503-511. (2000); Asakura et al., Genome 43:503-11 (2000); Ohtsuka et al., J. Fac. Agr. Hokkaido Univ. 65(2):127-98 (1991).)

One CMS method combined the cytoplasm of Triticum timopheevi, a wild relative of wheat, and the nuclear genomes of hexaploid wheat (having general genome composition AABBDD), or of triticale (having the general genome composition AABBRR). However, while cytoplasmic male sterility has been useful for controlled cross-pollination, male fertility has to be restored in the resulting hybrid seed and plants to enable commercial production of grain. The restoration genes have to be incorporated into male fertile pollinator lines, which then supply the pollen to the respective male sterile, female line during hybrid seed production. The Triticum timopheevi CMS method exhibits deficiencies of fertility restoration and restorer gene identification and has been abandoned by most breeding companies.

Another proposed method for CMS was to introduce cytoplasm from Aegilops squarrosa (Triticum tauschii) into a hexaploid wheat with a Triticum aestivum nuclear genome (AABBDD), and then seek a nuclear mutant that would control male fertility. The nuclear mutant would be induced by mutation. (See, e.g., U.S. Pat. No. 4,143,486.) The resulting alloplasmic plants, however, appeared to be male fertile, and the mutagenesis failed to identify a nuclear mutant that was incompatible with Ae. squarrosa cytoplasm. Thus, male sterility was not achieved, and the basis for the system was not realized (Maan, S. S., and Kianian, S., Personal communication, 2002).

In another method (see U.S. Pat. No. 4,680,888), cytoplasmic male sterility is manipulated by producing hybrid seed in an environment having no less than 14 hours day length. (See, e.g., Murai, Breeding Science 48:35-40 (1998); Murai, Euphytica 117:111-16 (2000).) Under these conditions, the plants are cytoplasmically-controlled male sterile; other, male fertile wheat plant provide the pollen for the hybrid seed production. Seed of the cytoplasmic male sterile, female plants is maintained by allowing selfing to occur in an environment with less than 14 hours day length. However, attempts to develop the system led to the conclusion that the system was not stable enough for successful commercial applications.

Methods of self-incompatibility for hybrid seed production have been reported for rye and oilseed rape, but not for wheat and triticale.

There remains a need, therefore, for systems and methods for producing hybrid wheat or triticale plants and seed.

### BRIEF SUMMARY OF THE INVENTION

The present invention relates to the production of polyploid, hybrid wheat plants and hybrid wheat seed via the employment of genetically-controlled cytoplasmic male sterility and genetically-controlled fertility and vigor restoration. The invention includes alloplasmic wheat plants having Aegilops squarrosa cytoplasm and recessive alleles of nuclear anther dehiscence-controlling Ad genes, which cause male sterility in certain hexaploid and tetraploid wheat plants having Ae. squarrosa cytoplasm. Male fertility is restored by a dominant Ad allele, common in most hexaploid wheat cultivars and lines, and in some tetraploid durum wheat cultivars and lines. Plant vigor and pollen viability in the alloplasmic plants (with Ae. squarrosa cytoplasm) are restored by two homoeologous genes, Cv and Cp.

In one aspect, polyploid, cytoplasmic male sterile (CMS), female wheat and triticale plants are provided. These plants generally has a genetic composition comprising group 1 chromosomes 1B″, or 1B″ and 1D″, and Aegilops squarrosa cytoplasm. The 1B″ and 1D″ chromosomes have Cv-(sq) and Cp-sq) alleles, which confer compatibility with Ae. squarrosa cytoplasm, and an inactive ad allele. In certain embodiments, the CMS wheat plants are hexaploid wheat having the genetic composition AAB″B″D″D″, or tetraploid wheat comprising the genetic composition AAB″B″, or triticale having the genetic composition AAB″B″RR. The CMS wheat plants are typically tolerant to an herbicide. In certain embodiments, seed of the CMS wheat or triticale plant is provided.

In a related aspect, polyploid, fertile male wheat and triticale plants are provided. These plants generally have a genetic composition comprising group 1 chromosomes 1B″, or 1B″ and 1D″, Triticum species cytoplasm, and resistance to a herbicide. In certain embodiments, the male fertile plants have the genetic composition AAB″B″D″D″, AAB″B″, or AA B″B″RR. The Triticum species cytoplasm can be, for example, Triticum aestivum L. cytoplasm. Seed from the male fertile plants is also provided.

In another aspect, a system is provided which includes CMS female polyploid wheat or triticale plants and polyploid, fertile male wheat or triticale plants. The fertile male plants have a genetic composition comprising group 1 chromosomes 1B″, or 1B″ and 1D″, Triticum species cytoplasm, and resistance to an herbicide. The CMS female plant and fertile male plant are tolerant to the same herbicide. The system can further include a pollen fertility restorer wheat plant comprising a dominant Ad allele, and Cp and Cv alleles.

In yet another aspect, polyploid, male sterile, female fertile wheat or triticale plants are provided, which have a general genetic composition of AABB, AABBDD, or AABBRR, and comprising Cv-(sq) and Cp-(sq) alleles and ad alleles in the B, or B and D genomes, respectively, and Aegilops squarrosa cytoplasm. These plants can be, for example, a hexaploid wheat comprising the genetic composition AAB″B″D″D″, a tetraploid wheat comprising the genetic composition AAB″B″, or a triticale comprising the genetic composition AAB″B″RR.

Methods of producing wheat and triticale seed are also provided. These methods generally include providing a polyploid, cytoplasmic male sterile (CMS), female wheat or triticale line having a genetic composition comprising group 1 chromosomes 1B″, or 1B″ and 1D″, Aegilops squarrosa cytoplasm, and tolerance to a herbicide. A maintainer line is also provided which has a genetic composition comprising group 1 chromosomes 1B″, or 1B″ and 1D″, and Triticum species cytoplasm. The CMS female plants are pollinated by the maintainer line, and the pollinated CMS female plants produce seed. The maintainer line is also tolerant to the herbicide. In certain embodiments, the CMS female line has the genetic composition AAB″B″D″D″, AA″B″ or AAB″B″RR. In additional embodiments, CMS female and maintainer lines carry Cp-(sq) and Cv-(sq) alleles, transferred directly or indirectly to their chromosomes 1A and 1B chromosomes from chromosomes 1A and 1G of Triticum timopheevi.

The method can further include growing seed from CMS female line to generate polyploid male sterile, female plants, and growing seed of a male fertile restorer line lacking tolerance to the herbicide to generate fertile male restorer plants. The CMS female line is pollinated by the restorer line. After pollination, the restorer line is treated with a herbicide to selectively kill the restorer line. Hybrid seed, when mature, can be harvested from the CMS female plants. In certain embodiments, the restorer line includes a dominant Ad allele on chromosome 1B, as derived from Triticum timopheevi, chromosome 1B of the durum variety Langdon, or from a durum plant carrying the Ad allele.

Tolerance to the herbicide can be, for example, induced by mutagenesis of the CMS female line, or the maintainer line, or introduced to the CMS female line, or maintainer line, by recombination breeding with a germplasm source carrying an induced herbicide tolerance mutation. In certain embodiments, the mutagenesis is performed by treatment of seed with a chemical or physical mutagen.

### BRIEF DESCRIPTION OF THE DRAWINGS

FIG. 1 depicts examples of analyses of the relative migration of gliadin proteins from gliadin loci by SDS gel electrophoresis. FIG. 1A depicts a comparison of the relative migration of gliadin proteins from the gliadin loci of chromosomes 1D′, 1B′ and 1B (lanes 4-6, respectively). Panel B depicts a comparison of the relative migration of gliadin proteins from the gliadin loci of chromosomes 1D′ and 1B′, 1B, 1D and 1D′ and 1B′ (lanes 3, 7, 8, and 13, respectively). The locations of the gliadin proteins are shown by brackets.

### DETAILED DESCRIPTION OF THE SPECIFIC EMBODIMENTS

The present invention provides methods and systems for cytoplasmically-controlled male sterility for hybrid seed production for wheat and triticale. In one aspect according to the present invention, cytoplasmically-controlled male sterility is provided in A line alloplasmic polyploid wheat or triticale plants. As used herein, the term “alloplasmic” refers to a plant that has a nucleus of a wheat line, cultivar or plant (e.g., from or derived from Triticum turgidum durum or Triticum aestivum L.) or a triticale line, cultivar or plant and an alien cytoplasm (e.g., from or derived from Aegilops squarrosa (Triticum tauschii)). Polyploid plants according to the present invention include two or more sets of chromosomes (e.g., tetraploids or hexaploids).

The alloplasmic plants according to the present invention also include alleles of a nuclear locus which controls anther dehiscence, the Ad locus. Alleles of the Ad locus are associated with cytoplasmic male sterility in alloplasmic wheat plants according to the present invention. The alloplasmic plants further include homeologous nuclear genes mediating vigor and protoplast restoration, the Cv and Cp genes, and at least one gene mediating tolerance to an herbicide. As used herein, the A line alloplasmic polyploid plants are also referred to as “male sterile, female” or “male sterile, female fertile” plants or lines.

In another aspect, B maintainer lines are provided which include euplasmic male fertile polyploid wheat or triticale plants having cytoplasm of, or derived from, a Triticum species (e.g., Triticum turgidum L. or Triticum aestivum L.) and recessive alleles of the Ad locus mediating male sterility in the presence of Ae. squarrosa cytoplasm. The B line plants also include alleles of the Cv and Cp genes mediating vigor and protoplast restoration, and at least one gene mediating tolerance to the herbicide to which the A line is highly tolerant.

In yet another aspect, restorer (R) lines are provided, which include euplasmic polyploid wheat and triticale plants having cytoplasm of a Triticum species (e.g., from or derived from Triticum turgidum L. or Triticum aestivum L.) and dominant alleles of the Ad locus mediating male fertility in the presence of Ae. squarrosa cytoplasm. The R line plants further include nuclear genes mediating vigor and protoplast restoration, the Cv and Cp genes, and are sensitive to the herbicide to which the A line is highly tolerant.

Male sterility in alloplasmic plants according to the present invention is effected by the Ad locus, which controls anther dehiscence. The Ad locus is located on the 1B chromosome (of the B genome) of durum wheat cultivars and lines, and the 1B and 1D chromosomes (of the B and D genomes) of hexaploid wheat cultivars and lines. Dominant Ad alleles 1D-Ad-(sq) and 1D-Ad-(eu) confer compatibility in alloplasmic plants having Ae. squarrosa cytoplasm. Recessive ad alleles (e.g., ad-(sq) and ad-(eu)) confer cytoplasmic male sterility in alloplasmic lines having Ae. squarrosa cytoplasm (e.g., when the nuclear genome is from, or derived from, T. turgidum durum or T. aestivum hexaploid wheat cultivars or lines). Recessive ad alleles according to the present invention prevent, or interfere with, anther dehiscence in alloplasmic wheat plants having Ae. squarrosa cytoplasm. These recessive ad alleles can include, for example, deletions of the Ad locus, as well as inactivating mutations of an Ad gene.

Plants according to the present invention further include two homoeologous genes, Cv and Cp, which restore or maintain plant vigor and pollen viability (protoplast restoration) in the presence of Ae. squarrosa cytoplasm. The Cv locus encodes a nuclear gene affecting plant vigor. The Cv-(sq) allele is compatible with Ae. squarrosa cytoplasm. The Cv-(eu) allele is compatible with euplasmic wheat cytoplasm (e.g., from AABB or AABBDD genome wheat plants), including Ae. squarrosa cytoplasm. The Cp locus encodes a nuclear gene affecting normal development or function of plastids (e.g., chloroplasts, amyloplasts). The Cp-(sq) allele is compatible with Ae. squarrosa cytoplasm. The Cp-(eu) allele is compatible with euplasmic wheat cytoplasm (e.g., from AABB or AABBDD genome wheat plants). The Cp-(sq) and Cv-(sq) alleles provide plant vigor and protoplast restoration in the presence of Ae. squarrosa cytoplasm. In the absence of the Cp-(sq) and Cv-(sq) alleles, seed from alloplasmic plants with Ae. squarrosa cytoplasm is abortive (e.g., inviable).

The Cv and Cp homeologous genes are present on the long arms of the 1A and 1G chromosomes of Triticum timopheevi (T. timopheevi var. typicum) (see, e.g., Asakura et al., Genome 43:503-11 (2000)). The 1G chromosome is closely related to the 1B chromosome of T. turgidum durum wheat. The homeologous Cv and Cp genes can be transferred from Triticum timopheevi (T. timopheevi var. typicum) to other wheat plants (e.g., T. turgidum durum wheat) by breeding or recombination methods. Such methods are disclosed in, for example, Asakura et al. (Genome 43:503-11 (2000)).

In additional embodiments, A line, male sterile, female plants also carry genes providing tolerance to an herbicide. As used herein, “tolerance” to an herbicide refers to an ability, trait or quality of a plant to withstand a particular herbicide at a dosage that is greater (usually substantially greater) than the dosage that other plants are able to withstand (e.g., herbicide-sensitive plants). Herbicide tolerance is typically dominant or semi-dominant. Herbicide tolerance can be present in one or more gene dosages, and in one or more genomes, depending on the degree of herbicide tolerance desired and the degree of tolerance conferred by each gene or allele. For example, one or more genomes can be homozygous for an allele(s) conferring tolerance to the herbicide. In certain embodiments, high herbicide tolerance is provided by multiple dominant or semi-dominant alleles in the genomes (chromosome sets), which facilitates selective destruction of an herbicide-susceptible male parent by herbicide treatment after pollination has occurred. High levels of herbicide tolerance can also facilitate weed control in the hybrid crop.

In exemplary embodiments, the A lines (cytoplasmic male sterile) and B lines (maintainer) according to the present invention include the 1B″, or 1B″ and 1D″ chromosomes, in euploid (i.e., having full chromosome sets) tetraploid and hexaploid plants, respectively. As used herein, the terms “1B″ chromosome” and “1D″ chromosome” refer to chromosomes having an inactive Ad allele (e.g., a deletion or inactivation) and Cv-(sq) and Cp-(sq) alleles. The term “1D′ chromosome” refers to a ID chromosome having a deletion of at least a portion of the Ad locus. A “B′” genome is a B genome having a “1B′” chromosome. A “D′” genome is a D genome having a “1D′” chromosome.

A typical hexaploid A line (cytoplasmic male sterile) according to the present invention has the genetic constitution (sq)AAB″B″D″D″, where (sq) denotes Ae. squarrosa cytoplasm; B″ indicates a B genome having a 1B″ chromosome, which lacks a dominant Ad allele, or carries a recessive ad allele, and includes the Cv-(sq) and Cp-(sq) alleles; and D″ indicates a D genome having a 1D″ chromosome, which lacks a dominant Ad allele, or carries a recessive ad allele, and includes the Cv-(sq) and Cp-(sq) alleles. A typical tetraploid (durum) A line (cytoplasmic male sterile) according to the present invention has the genetic constitution (sq)AAB″B″, where (sq) denotes Ae. squarrosa cytoplasm and B″ indicates a B genome having a 1B″ chromosome, which lacks a dominant Ad allele, or carries a recessive ad allele, and includes the Cv-(sq) and Cp-(sq) alleles. To achieve compatibility with the Ae. squarrosa cytoplasm, the 1A and 1B or 1B′ chromosomes of durums can be bred to include the Cv-(sq) and Cp-(sq) alleles, derived from T. timopheevi, while the 1D chromosomes of most common hexaploid wheats, and those bred to carry the 1D′ chromosome, typically carry also Cv-(sq) and Cp-(sq).

In a specific embodiment, the 1D′ chromosome has a deletion at the tip of the short arm (distal to the Gli-1 gene at about −35.8 cM), which includes a deletion of at least a portion of the Ad locus. A 1D′ chromosome that retains the Cv-(sq) and Cp-(sq) alleles is also referred to as a 1D″ chromosome. The 1D′ chromosome was originally derived from the genetically similar D genome of Ae. squarrosa var. typica (the cytoplasmic donor). In contrast, the typical ID chromosome of hexaploid wheat cultivars and lines includes the Cv and Cp loci, as well as a dominant Ad allele, which confers male fertility in alloplasmic lines.

In additional embodiments, A lines (cytoplasmic male sterile) and B lines (maintainer) of triticale are provided. A typical hexaploid A line (cytoplasmic male sterile) according to the present invention has the genetic constitution (sq)AAB″B″RR, where (sq) denotes Ae. squarrosa cytoplasm; and B″ indicates a B genome having a 1B″ chromosome, which lacks a dominant Ad allele, or carries a recessive ad allele, and includes the Cv-(sq) and Cp-(sq) alleles. In certain embodiments, triticale A and B lines according to the present invention can include a 1BL/IRS or 1DL/RS translocation(s), such as is disclosed in, for example, Lukaszewski, Crop. Sci. 40:216-25 (2000), and Lukaszewski, Crop. Sci. 41:1062-65 (2001)(the disclosures of which are incorporated by reference herein). In addition, alleles of the Ad locus can be bred into triticale, such as by recombination with a triticale line having a 1BL/lRS or 1DL/IRS translocation. Methods similar to those used for wheat breeding and recombination, as exemplified herein or as known in the art, can be used to make A and B triticale lines according to the present invention. (See, e.g., Lukaszewski (2000), supra; Lukaszewski (2001), supra; Lukaszewski et al., Crop Sci. 41:1743-49 (2001); the disclosures of which are incorporated by reference herein).

In exemplary embodiments, A lines and B lines according to the present invention can be constructed, for example, by backcrossing a wheat line to introduce the 1B″, or 1B″ and 1D″ (or chromosomes (including the Cv-(sq), Cp-(sq) and ad alleles), into wheat lines having Ae. squarrosa cytoplasm. The resulting wheat plants will carry the T. timopheevi-derived Cv-(sq) and Cp-(sq) alleles on their 1A and 1B″ chromosomes (for the male sterile, female A line, and the B maintainer lines, as derived from T. timpheevi). Such backcrossing can be performed by breeding and recombination methods known to the skilled artisan. In certain embodiments, the presence of the 1D″ chromosomes can be determined by monitoring the presence (or absence) of gliadin protein Gli-D1, which locus is tightly associated with the nuclear male sterility-facilitating Ad genes, active in the presence of Ae. squarrosa cytoplasm (infra). Alternatively, the Cv(sq), Cp-(sq) and ad alleles can be introduced by recombinant DNA techniques.

The incorporation of the Cv-(sq) and Cp-(sq) alleles into durums with Ae. squarrosa cytoplasm via genetic recombination is straightforward, because the Cv and Cp alleles confer vigor and pollen viability to the plants recovered. For example, the Cv and Cp alleles can be transferred to the A and B genomes of NPB871104 (via the construction AAB′B′+1D′) by standard breeding and recombination techniques, and the introduction of Ae. squarrosa cytoplasm. The constructed plant, (sq)NPB871104+1D′, which is male sterile, can be bred with T. timopheevi var. typicum to recover (sq)AAB″B″ male sterile progeny carrying the Cv and Cp genes transferred from T. timopheevi. Viable seed from this cross can be recovered and can include the Cv-(sq) and Cp-(sq) alleles and Ad or ad alleles. Plants carrying the ad allele will be male sterile. The Cv-(sq) and Cp-(sq) alleles also can be recombined into other genetic backgrounds for developing durum parents by methods which are familiar to plant breeders. In another exemplary embodiment, introduction of herbicide tolerance genes into durum (e.g., NPB871104), which has T. turgidum durum cytoplasm can be accomplished along with introducing the Cp-(sq) and Cv-(sq) alleles to develop the maintainer stock for the male sterile.

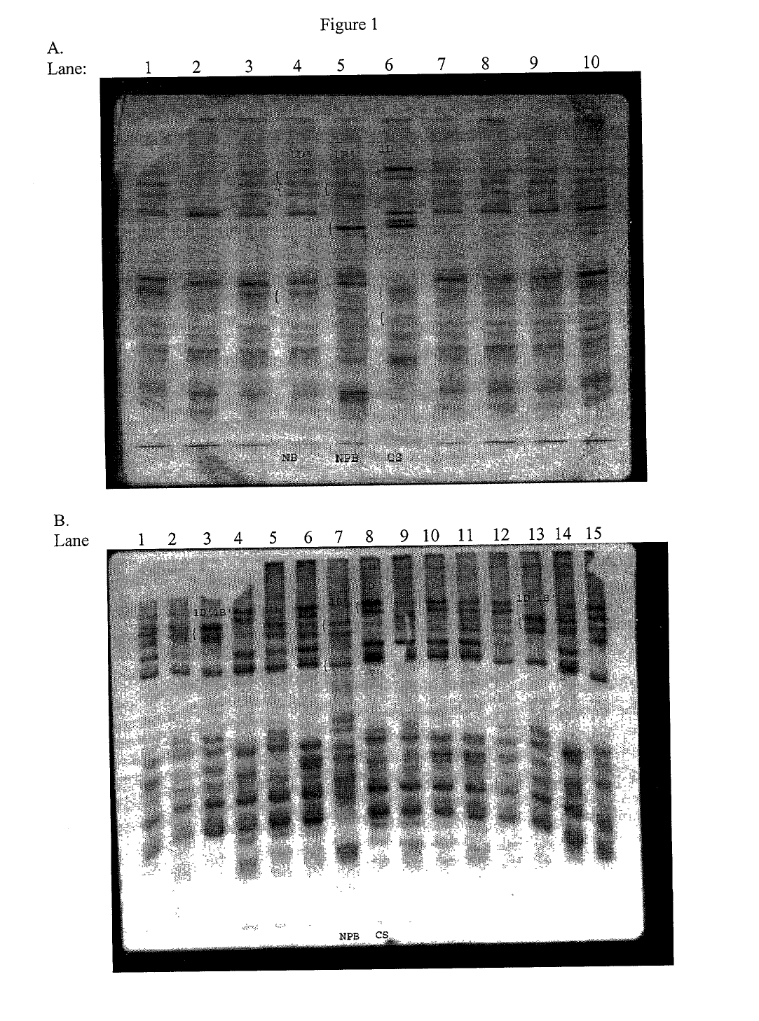

The presence of recessive ad alleles in wheat and triticale plants can be detected visually in flowering wheat/durum spikes by noting the small, deformed/indehiscent anthers that do not extrude from the glumes of cytoplasmic male sterile plant spikes. In addition, the Ad-(sq) gene on the 1D chromosome is closely linked to the Gli-1 locus. The Gli-1 proteins (produced by the Gli-D1 gene) are distinguishable by protein electrophoresis techniques (e.g., SDS PAGE, isoelectric focusing, 2-dimensional electrophoresis, and the like) from the group 1 chromosome ID. For example, the Gli-1 proteins (produced by the Gli-D1 gene) are distinguishable by SDS polyacrylamide gel electrophoresis (SDS PAGE) from the gliadin protein bands controlled by the Gli-1 gene on the ID chromosome (e.g., of Triticum aestivum cv. Chinese Spring or other bread wheats). (See FIG. 1.) (See also, e.g., Metakovsky, J. Genet. & Breed, 45:325-44 (1991).) Thus, inheritance of ad alleles (e.g., on the 1D′ or 1D″ chromosome) can be followed and/or confirmed by the presence of Gli-D1 proteins.

As noted above, the 1B″ chromosome also contains an ad allele for male sterility in durums carrying Ae. squarrosa cytoplasm. Transfer of the 1B″ chromosome can be detected by following the gliadin loci on this chromosome or other genetic markers on the 1B chromosome. The 1B chromosome has two gliadin protein loci on its short arm, at positions −49.0 cM (Gli-B1) and −20.1 (Glul-B3). The 1B″ chromosome also includes the Cp-(sq)and Cv-(sq) alleles. The Cp-(sq)and Cv-(sq) alleles can be transferred to the 1B or 1B′ chromosomes from the 1A and 1G chromosomes of T. timopheevi (e.g., T. timopheevi var. typicum, Asakura et al. (2000), supra) by standard breeding or recombination methods. In certain embodiments, the 1B″ chromosome can be derived from Northwest Plant Breeding Co. T durum selection, NPB871104, or genetically-related lines or cultivars.

The recovery of recombinant lines carrying a recessive ad allele according to the present invention on the 1B″ chromosome also can be achieved by selection for lack of anther dehiscence in double haploid (DH) or F2 durum progenies with (sq) cytoplasm and homoeologous genes Cv-(sq) and Cp-(sq) on 1A and 1B″, as transferred from T. timopheevi. (See, e.g., Asakura et al. (2000), supra; U.S. Pat. No. 6,362,393; the disclosures of which are incorporated by reference herein.) In additional embodiments, the presence of the cytoplasmic male sterility-controlling ad genes can be determined by ELISA, or other immunoassay, or marker-assisted techniques, such as RFLP mapping, RAPD marker mapping, allele-specific PCR, and the like. (See, e.g., Asakura et al., Genome 40:201-10 (1997); Giersch et al., Cereal Chemistry 76:380-88 (1999); Nicolas et al., Food and Agricultural Immunology 12:53-65 (2000); Verity et al., Cereal Chemistry 76:673-81 (1999).) (See generally Harlow and Lane, Using Antibodies, A Laboratory Manual, Cold Spring Harbor Laboratory, New York (1999); Sambrook et al., Molecular Cloning, A Laboratory Manual, 3rd ed., Cold Spring Harbor Publish, Cold Spring Harbor, N.Y. (2001); Ausubel et al., Current Protocols in Molecular Biology, 4th ed., John Wiley and Sons, New York (1999); U.S. Pat. Nos. 4,683,202, 4,683,195 and 4,800,159; Innis et al., PCR Protocols: A Guide to Methods and Applications, Academic Press, Inc., San Diego, Calif. (1989); Innis et al., PCR Applications: Protocols for Functional Genomics, Academic Press, Inc., San Diego, Calif. (1999); White (ed.), PCR Cloning Protocols: From Molecular Cloning to Genetic Engineering, Humana Press, (1996); EP 320 308; the disclosures of which are incorporated by reference herein in their entirety).

A hexaploid maintainer (B line) according to the present invention can be constructed by backcrossing a wheat line to introduce the 1B″ and 1D″ chromosomes into a hexaploid wheat carrying T. aestivum L. cytoplasm. A typical hexaploid B maintainer line has the genetic constitution (eu)AAB″B″D″D″, where (eu) denotes euplasmic wheat cytoplasm; B″ indicates a B genome having a 1B″ chromosome but without a dominant Ad allele; and D″ indicates a D genome having a 1D″ chromosome (e.g., with Cv-(sq), Cp(sq), but without a dominant Ad allele). The maintainer (B) line typically has T. aestivum L. cytoplasm, allowing seed reproduction of the B lines by selling. The maintainer B line can also include tolerance to the same herbicide as the A line, thereby maintaining the genetic basis for herbicide tolerance in the A lines.

The B lines provide the pollen source for maintaining and increasing seed of the A line stocks. The A and B lines are typically grown separately, but in sufficient proximity (e.g., as in separate strips planted nearby each other), or with the A line surrounded by the B line, to allow wind-aided pollination of the A line by the B line to increase the quantity of the A line seed stocks, as needed for the production of hybrid seed (e.g., for commercial sale). Since the proportion of cytoplasmic male sterile (A line) to be reproduced will be appreciably less than that used in the production of hybrid seed, the increased cost of cytosterile seed stock due to the necessity for separation of the parent A and B lines may not be a significant limitation. Thus, it can be less efficient, but feasible economically, to reproduce A line seed in a manner similar to that previously employed for CHA and other CMS systems, such as by planting the A line either in strips between plantings of the B line pollen-providing parent. The seeds of each line can be harvested separately.

Restorer (R) lines generally include hexaploid wheats carrying the 1D chromosome which carry the Cp, Cv and Ad alleles, which provide for normal plastid development, for plant vigor, and for fertility restoration (anther dehiscence). Nearly all hexaploid wheat plants may carry Ad alleles on their 1D chromosomes, thus can act as male parent, ‘R’ (fertility restorer) lines. Similarly, tetraploid durum wheats can include, or can be bred to include, the Cv, Cp and Ad alleles on their 1A and 1B chromosomes, and can also be male parent, R lines. Because some durum wheats already may carry the chromosome 1B fertility restorer Ad gene of Ae. squarrosa cytoplasmic sterility, effective pollinators can be bred or selected by, for example, interbreeding or combining such wheats.

R lines also can be used to introduce new traits into the A lines and B lines, usually via the maintainer lines. For example, wheat plants developed by breeders can be used as the male parents for hybrids, if the flour quality, agronomic and disease resistance traits are favorable, expanding the potential germplasm base for available for F1 hybrid production.

The typical cytoplasmic male sterile A lines, and maintainer B lines, each carry herbicide tolerance genes in the same two genomes. This allows the F1 hybrids to possess multiple doses of herbicide tolerance gene alleles, providing a mechanism for destroying the seed-producing ability of the male (R) parent, which lacks the herbicide tolerance of the A line and B line parents. The male parent is typically treated with herbicide (e.g., spraying) after pollination of the male sterile line has occurred. The herbicide destroys the male (R), non-tolerant plants, or cause them to be infertile. The herbicide typically allows rapid killing, or induction of inviable seed (e.g., within about 3 days after herbicide exposure), of non-tolerant (R) male (i.e., pollen-providing) adult plants after pollination has been completed. Because seed of the non-tolerant male plant are inviable, there is no need to sort seeds from the male and male sterile female (A line) parents; the seed of the A lines can be mixed with the seed of the non-tolerant male lines for F1 hybrid wheat seed production. In various embodiments, essentially 100% hybrid seed can be produced and harvested. The F1 hybrids typically also have sufficient additive herbicide tolerance, via multiple heterozygous herbicide tolerance genes, for controlling weeds among the F1 hybrid plants, when grown in the field.

A line seed can be mixed with the seed of the non-tolerant R male lines at planting. The proportions of female (A line) to male fertile (R line) stock seed sown for commercial hybrid seed production can be as low as, for example, 90-85% to 10-15%.

Representative herbicidal compounds to which herbicide tolerance can be induced, or incorporated by breeding, in the male sterile A lines include, for example, imidazolinones (e.g., imazamox, and similar compounds), or cyclohexenones (e.g., sethoxydim, BAS620H, etc.), and the like. Imazamox-tolerant durums and common hexaploid wheats have been induced, and are available for recombination breeding. Generally, imidazolinone tolerance in the male sterile A lines is present in the A and B genomes of tetraploids, or A and B, A and D, B and D, or A, B and D or R genomes of hexaploids (including triticales), in order that the F1 hybrid progeny can carry sufficient tolerance for weed control in the field. For other herbicide tolerance, the number of herbicide tolerance genes present can vary, depending on the level of tolerance provided by each gene.

Herbicide tolerance can be introduced into wheat and triticale plants, for example, by transfer of herbicide tolerance genes from herbicide tolerant germplasm stocks by breeding, by recombinant DNA techniques, and/or by mutagenesis of maintainer wheat lines. Suitable target wheat lines include, but are not limited to T. aestivum and T. turgidum durum wheats. Methods for breeding wheat and triticale are well known in the art. In addition, mutations for herbicide tolerance in hexaploid and durum wheat can be induced, such as, for example, mutations for imazamox tolerance. Herbicide tolerance genes induced in durum wheats can be readily transferred to triticale strains by genetic recombination methods familiar to those experienced in the art.

In an exemplary embodiment, a wheat plant, or parts thereof, can be mutagenized with any of several known mutagens, and herbicide tolerance mutants recovered from among M2 generation field grown seedlings. In certain embodiments, the seed is treated with mutagen(s). The amount of seed to be mutagenized can be selected according to the desired number of “hits” in the genome(s), the screening efficiency, and the like. The mutagens can be, for example, chemical or physical mutagens. Suitable chemical mutagenizing agents include, but are not limited to, ethyl methanesulfonate (EMS), diethyl sulfate, or EMS, followed by azide (e.g., sodium or potassium) treatment (see, e.g., co-pending U.S. patent application Ser. No. 09/719,880, filed Dec. 18, 2000; International Patent Publication WO 99/65292; the disclosures of which are incorporated by reference herein), nitrosoguanidine, N-methyl nitrosourea, N-diethyl nitrosourea, or other alkylating agents, and physical agents, such as electromagnetic radiation, X-rays, gamma rays, thermal or fast neutrons, and the like. Combinations of mutagens, either chemical and/or physical, can be employed.

As will be appreciated by the skilled artisan, other mutagenic agents can also be used. (See also Konzak et al., Mutation Breeding Manual, 2^{nd} ed., International Atomic Energy Agency, Tech. Reports Series 119 (1977), the disclosure of which is incorporated by reference herein). Following mutagenesis, the treated seeds are planted and the M1 generation plants are grown to produce M2 (second generation) seed. Plants grown from such seed are screened for herbicide tolerance by spraying the M2 generation seedlings or plants, with appropriate doses of the herbicide, selecting and growing to maturity those seedlings or plants surviving the herbicide treatment, and reevaluating the level of induced herbicide tolerance of the mutants by progeny testing, according to methods known in the art.

In another aspect, methods of producing wheat and triticale seed are provided. For example, in certain embodiments, seed from an A line (polyploid, cytoplasmic male sterile, female fertile wheat line) is provided. Generally, the A line has the genetic composition AABB or AABBDD and includes the group 1B″, or 1B″ and 1D″ chromosomes, Aegilops squarrosa cytoplasm, and tolerance to an herbicide. For example, the A line can have the genetic composition (sq)AAB″B″ or (sq)AAB″B″D″D″ or (sq)AAB″B″RR.

A B maintainer line is also provided. The B line generally has the general genetic composition (eu)AABB, (eu)AABBDD or (eu)AABBRR (according to the genetic composition of the A line), and includes the group 1B″, or 1B″ and 1D″ chromosomes, and Triticum species cytoplasm. The B line is also typically tolerant to the same herbicide as the A line. The B line can have the genetic composition, for example (eu)AAB″B″, (eu)AAB″B″D″D″ or (eu)AAB″B″RR.

The A and B lines can be grown, for example, in the separate, machine-harvestable adjacent rows or strips, or by surrounding the A line plants with the B line plants, or interspersed with each other. The A line is pollinated by pollen from the B maintainer line, typically by wind, although other methods are possible and within the scope of the invention. A line, or progeny, seed can then be collected from the pollinated A line. Depending on the genetic composition of the A and B lines, the resulting seed can be A line seed, or hybrid seed.

In additional embodiments, A line or progeny seed can be grown to generate polyploid male sterile, female fertile plants. A male fertile, restorer (R) line is also grown. The restorer line typically includes a dominant Ad allele, but is sensitive to the herbicide. In certain embodiments, A line and R line seed are planted in the same plot, such as by mixing the seed prior to planting. In other embodiments, the A lines and R lines are planted in separate, adjacent rows or by surrounding the A line plants with the R line plants. Pollen from the R line plants is allowed to pollinate the A. Following pollination, the R line is contacted with the herbicide (to which the A line is tolerant) to kill the R (e.g., to kill the plants, or to prevent the formation of seed by the R line, and the like). The herbicide is typically contacted with the R line by spraying, although other methods are possible and within the scope of the invention. The seed can then be harvested or collected from the pollinated A line, when mature, as desired.

### EXAMPLES

The following examples are provided merely as illustrative of various aspects of the invention and shall not be construed to limit the invention in any way.

### Example 1

An alloplasmic cytoplasmically male sterile (CMS), female line, with Ae. squarrosa cytoplasm, is established as follows, using the gliadin protein and other markers for guiding the transfer of an ad allele. The transfer of the 1B′ and 1D′ chromosomes between lines is followed using protein makers for the gliadin proteins of endosperm. The genes for gliadin proteins, genes Gli, are located on the short arms of the homoeologous 1 and 6 group chromosomes in tetraploid and hexaploid wheat. The 1D chromosome has one Gli locus Gli-1 at about −35.8 cM, which is linked to the Ad locus. The 1B chromosome has two Gli loci, Gli-B1, at about −48.0 cM and Gli-B3 at about −20.1 cM, which can be used to follow the 1B′ chromosome. The presence of the Ad locus is separately confirmed, using other markers. The gliadin proteins of the 1B′ and 1D′ chromosomes are distinguishable from those of the 1B and 1D chromosomes of T. aestivum cv. Chinese Spring and other bread wheats by SDS gel electrophoresis.

(1) The 1B′ and 1D′ chromosomes are introduced into euplasmic (eu) and alloplasmic, Ae. squarrosa (Triticum tauschii) (sq), cytoplasm bread wheat. A durum construction (CMS-(sq)AAB′B′+1D′) is crossed by a euplasmic wheat ((eu) AABBDD (e.g., T. aestivum cv. Chinese spring or Pi 574537) as follows:

CMS-(sq)AAB′B′+1D′)×(eu)AABBDD (e.g., (Pi574537 or Chinese Spring) Of the resulting F1 individuals, some have the composition (sq)AABB′D (14II+7I, 2n=35) and have anthers that dehisce normally due to the presence of the Ad-1B and Ad-1D alleles on 1B and 1D from the male parent. The pollen fertility will be normal, however, because one chromosome (1D) carries a Cp-1D and a Cv-1D gene, and an Ad gene. These F1 individuals without 1D′ are discarded, based on the gliadin analysis.

Other F1 individuals had the composition (sq)AABB″D+1D′ (15II+6I, 2n=38) and had anthers that dehisce normally due to the presence of the Ad-1B and Ad-1D alleles on the 1B and 1D chromosomes from the male parent. Pollen fertility will be high due to the presence of Ad allele (on the 1B and 1D chromosomes), and the plants will be viable and vigor due to Cp- and Cv-1D genes on the 1D chromosome, and the 1D″ chromosome from the male and female parents, respectively. DH or F2 plants having the 1D′ chromosome are identified by selecting for male sterility/lack of anther dehiscence, and by analyses for the absence of the gliadin locus on the 1D′ chromosome by SDS gel electrophoresis. (See, e.g., Metakovsky, J. Genet. & Breed, 45:325-44 (1991).)

Then, a backcross of the F2 (or DH) male sterile, CMS(sq)AAB′B′D′D′ is made to the AABBDD parent to recover a male sterile BC1 F2 or DH, and a second backcross is made, repeating the procedure to recover essentially a male sterile plant with the genes of the male parent, thus to recover a hexaploid A line with the genes of the male parent line. The maintainer B line also can be recovered from the same initial cross. A fertile DH or F2 plant is crossed back to the recurrent parent hexaploid line to place the nuclei with ad alleles of the 1B″ and 1D″ chromosomes into (eu) cytoplasm. Analyses for the Gli-1D′ locus will allow selection for the ad allele. The 1B ad allele can be identified by a DNA marker analysis. Alternatively, selection by 1B gliadin proteins can be employed to identify the 1B″ chromosome present in some of the progeny. Then a test cross to the male sterile A line would identify a B line maintainer based on the recovery of F1 male sterile progeny from the test cross.

(2) A (sq) durum wheat construction, AABB+1D′, is crossed with F1 individuals (from (1) above) having the genetic composition (sq)AABB′D+1D′ (15II+8I, 2n=38). The cross is as follows:

(sq)AAB′B′+ID′×F1 (sq)AABB′DD′ (fertile F1)

The F1 from this cross will have variable chromosome numbers (2n=36 to about 2n=42). Progeny individuals are selected having the composition (sq)AABB′DD′ (2n=42) among the second F1 ((sq)B1-F1) plants using the gliadin protein markers for transfer of the ad allele on chromosome 1D′. These selected individuals are allowed to self-pollinate, or DH are produced, to identify cytoplasmically male sterile, female fertile plants.

From amongst the next generation of F2 ((sq)B1-F2), cytoplasmically male sterile individuals of the (sq)AAB″B″D″D″ genetic composition, with no anther dehiscence and with ad alleles not compatible with Ae. squarrosa cytoplasm, are identified. The identification of cytoplasmically male sterile individuals of the (sq)AAB′B′D′D′ genetic composition can be confirmed by for example, crossing the F2 individuals to a tester line.

### Example 2

A cytoplasmic male sterile, female wheat line can be bred with a desired cultivar of bread wheat. The genetic constitution of the F₁ hybrids (by the natural pollination) will be as follows:

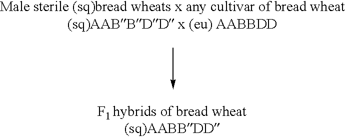

The F1 hybrids have the Ad-1B and Ad-1D alleles, two alleles of the Cp-1D gene and two alleles of the Cv-1D gene, all of which are compatible with the cytoplasm of Ae. squarrosa. The F₁ hybrids of bread wheat, (sq)AABB″DD″, will generally have the following characteristics:

(a) Anther dehiscence is generally normal because the F₁ hybrids have two Ad gene alleles compatible with Ae. squarrosa cytoplasm, the Ad-1B gene of the 1B chromosome 1B and the Ad-1D gene of the 1D chromosome.

(b) Pollen fertility is generally normal (e.g., eliminating or minimizing negative effects of the Ae. squarrosa cytoplasm) because the F₁ hybrids have the Cp-1D alleles which are compatible with Ae. squarrosa cytoplasm, located on both the 1D and 1D″ chromosomes.

(c) Plant growth and plant vigor are generally (e.g., eliminating or minimizing negative effects of the Ae. squarrosa cytoplasm), due to the presence of Cp-1D gene alleles on the 1D and 1D″ chromosomes, and the Cv-1D gene alleles located on the 1D and 1D″ chromosomes.

### Example 3

Triticale male steriles can be bred using the T. turgidium line derivative from the T. timopheevi cross as a female line carrying Cv and Cp genes in its 1A and 1B chromosomes in a CMS (sq)AAB″B″ parent, to cross with the male parent triticale (eu)AABBRR.

Male sterile segregants or DH can be recovered in the F2 or in 1 generation via the DH technology, even though the F1 will be a pentaploid, and as some of the AABB parental lines of the triticales carry no Ad gene, nor the Cv and Cp genes. The F2 plants and DH recovered will be those carrying the Cv and Cp genes. Those plants with the Ad allele will be fertile, those with the ad allele will be male sterile. If male sterile, the MS pentaploid can be backcrossed to the triticale parent to recover a stabilized male sterile line of the genetic structure of that triticale genotype. If the triticale does carry the ad allele, then it can be used to develop a male fertile, restorer line by crossing and backcrossing to a T. turgidem R line with (eu) cytoplasm, and the Ad, Cv and Cp genes. If the T. turgidem fertile line carries herbicide tolerance, tolerance can be selected for among F2 progeny, or in DH culture of germinating ambryoids.

The only progeny recovered from this pentaploid and backcrosses will be those with the Cv and Cp genes on their 1A and 1B chromosomes. Once recovered, these lines can be used as testers against the CMS triticale lines, developing a potential family of triticale (R) line restorers. However, as some triticales may already carry an Ad allele and can be converted to R. restorers by incorporating the Cv and Cp genes, either from the CMS male sterile lines or from test crosses with a CMS T. turgidum line.

### Example 4

Male sterile durum wheat A lines can be produced by crossing the (sq)AAB′B′+1D′ by T. timopheevi, producing DH or F1 plants from the cross will yield MS (sq)AAB′B plants carrying the Cv and Cp genes on their 1A and 1B chromosomes as transferred from T. timopheevi. Once a MS (sq)AAB″B″ plant is recovered, a maintainer line is developed by crossing the MS (sq)AAB″B″ plant with a normal durum plant (e.g., having a normal genotype), which may carry Ad allele, to produce a fertile F1, from which the F2 or DH are produced to recover MS (sq)AAB″B″ plants and F(fertile) (sq)AABB(with Ad) are obtained. After 3-4 backcrosses, the durum A (male sterile) line (sq)AAB″B″ plants and maintainer plants are recovered, by crossing an F1 of a later generation backcross to a (eu)AABB plant from which F2 or DH are recovered. The F2 or DH are used as testers against the MS(sq)AAB″B″ plants. An F2 fertile plant or DH with (eu) cytoplasm, which produces MS F1 cross progeny with the (sq) AAB″B″ backcross F2 plants can be the B line maintainer for the A line. The B line can reproduced by selling to continue the line.

The previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

What is claimed is:

1. A polyploid, cytoplasmically male sterile, female plant having a genetic composition comprising group 1 chromosomes 1B″, or 1B″ and 1D″, and Aegilops squarrosa cytoplasm.

2. The polyploid plant of claim 1, which is a hexaploid wheat comprising the genetic composition AAB″B″D″D″.

3. The polyploid plant of claim 1, which is a tetraploid wheat comprising the genetic composition AAB″B″.

4. The polyploid plant of claim 1, which is a triticale comprising the genetic composition AAB″B″RR.

5. The polyploid plant of claim 1, which is tolerant to an herbicide.

6. Seed derived from the polyploid plant of claims 1.

7. A polyploid, fertile male plant having a genetic composition comprising group 1 chromosomes 1B″, or 1B″ and 1D″, Triticum species cytoplasm, and resistance to a herbicide.

8. The polyploid plant of claim 7, comprising the genetic composition AAB″B″D″D″.

9. The polyploid plant of claim 7, comprising the genetic composition AAB″B″.

10. The polyploid plant of claim 1, comprising the genetic composition AAB″B″RR.

11. The polyploid plant of claim 7, which has Triticum aestivum L. cytoplasm.

12. Seed derived from the polyploid plant of claims 7.

13. A system comprising the polyploid plant of claim 5, and further comprising: a polyploid, fertile male plant having a genetic composition comprising group 1 chromosomes 1B″, or 1B″ and 1D″, Triticum species cytoplasm, and resistance to a herbicide; wherein the cytoplasmically male sterile, female plant and the fertile male plant are tolerant to the same herbicide.

14. The system of claim 13, further comprising a pollen fertility restorer plant comprising a dominant Ad allele, and Cp-(sq) and Cv-(sq) alleles.

15. A polyploid, male sterile, female wheat plant having a general genetic composition of AABB or AABBDD, and comprising Cv-(sq), Cp-(sq) alleles and ad alleles in the B, or B and D genomes, respectively, and Aegilops squarrosa cytoplasm.

16. The polyploid plant of claim 15, which is a hexaploid wheat comprising the genetic composition AAB″B″D″D″.

17. The polyploid plant of claim 15, which is a tetraploid wheat comprising the genetic composition AAB″B″.

18. A method of producing seed, comprising:
(a) providing a polyploid, cytoplasmic male sterile, female line having a genetic composition comprising group 1 chromosomes 1B″, or 1B″ and 1D″, Aegilops squarrosa cytoplasm, and tolerance to a herbicide;
(b) providing a maintainer line having a genetic composition comprising group 1 chromosomes 1B″, or 1B″ and 1D″, and Triticum species cytoplasm;
(c) pollinating the polyploid, cytoplasmic male sterile, female line with pollen from the maintainer line; and
(d) allowing the pollinated polyploid, cytoplasmic male sterile, female line to produce seed.

19. The method of claim 18, wherein the maintainer line is tolerant to the herbicide.

20. The method of claim 19, wherein the tolerance to the herbicide is induced by mutagenesis of the polyploid, cytoplasmic male sterile, female line or the maintainer line, or is introduced to the cytoplasmic male sterile, female line and maintainer line by recombination breeding with a germplasm source carrying an induced herbicide tolerance mutation.

21. The method of claim 20, wherein the mutagenesis is by treatment of seed with a chemical or physical mutagen.

22. The method of claim 18, wherein the pollination is by wind.

23. The method of claim 18, wherein the polyploid cytoplasmic male sterile, female line has the genetic composition AAB″B″D″D″.

24. The method of claim 18, wherein the polyploid cytoplasmic male sterile, female line has the genetic composition AAB″B″.

25. The method of claim 18, wherein the cytoplasmic male sterile, and maintainer lines carry Cp-(sq) and Cv-(sq) alleles, transferred to their chromosomes 1A and 1B chromosomes from chromosomes 1A and 1G of Triticum timopheevi.

26. The method of claim 18, further comprising:
(e) growing the seed from (d) to generate polyploid male sterile, female plants;
(f) growing seed of a male fertile restorer line lacking tolerance to the herbicide;
(f) pollinating the polyploid male sterile, female plants of (e) with pollen from the restorer line;
(g) contacting the restorer line with the herbicide to selectively kill the restorer line; and
(h) harvesting hybrid seed from the polyploid male sterile, female plants.

27. The method of claim 26, wherein the restorer line includes a dominant Ad allele on chromosome 1B, as derived from Triticum timopheevi, chromosome 1B of the durum variety Langdon, or from a durum plant carrying the Ad allele.

28. The method of claim 18, wherein the polyploid cytoplasmic male sterile female line is a triticale line, which has the genetic composition (sq)AAB″B″RR.

29. The method of claim 28, wherein the maintainer is a triticale maintainer line.

30. The method of claim 26, wherein the restorer line is a triticale line restorer line which carries 1A and 1B genes Cv and Cp and a 1B gene Ad for anther dehiscence.
